(19) United States
(12) Patent Application Publication
(10) Pub. No.: US 2010/0292527 A1
(43) Pub. Date: Nov. 18, 2010

(54) DEVICE AND METHOD FOR HYPERTENSION TREATMENT BY NON-INVASIVE STIMULATION TO VASCULAR BARORECEPTORS

(76) Inventors: M. Bret Schneider, Portola Valley, CA (US); Michael J. Partsch, Redwood City, CA (US); David J. Mishelevich, Playa del Rey, CA (US)

Correspondence Address: SHAY GLENN LLP, 2755 CAMPUS DRIVE, SUITE 210, SAN MATEO, CA, 94403, US

(21) Appl. No.: 12/671,141

(22) PCT Filed: Jul. 30, 2008
(86) PCT No.: PCT/US2008/071664

§ 371 Date: Jun. 17, 2010

Related U.S. Application Data
(60) Provisional application No. 60/953,191, filed on Jul. 31, 2007.
(60) Provisional application No. 61/018,449, filed on Jan. 1, 2008.

(51) Int. Cl. A61N 1/36; A61H 1/00; A61N 7/00; A61N 2/00

(52) U.S. Cl. 600/15; 607/44; 601/47; 601/2; 601/46

(57) ABSTRACT

The treatment of hypertension may be accomplished by stimulation of the carotid baroreceptors. In the present application the inventors disclose methods in which non-invasively-delivered mechanical perturbations caused by sound, ultrasound, or electrical perturbations caused by magnetic, or direct current stimulation may be used to stimulate the carotid baroreceptors, triggering physiological responses that treat medical disorders including hypertension.

### CROSS REFERENCE TO RELATED APPLICATIONS

This application claims priority to U.S. Provisional Patent Application Ser. No. 60/953,191, filed on Jul. 31, 2007, titled “DEVICE AND METHOD FOR TREATING HYPERTENSION VIA NON-INVASIVE NEUROMODULATION”, and U.S. Provisional Patent Application Ser. No. 61/018,449, filed on Jan., 1, 2008 entitled “DEVICE AND METHOD FOR TREATING HYPERTENSION BY SONIC STIMULATION AND DIRECT ELECTRICAL CURRENT TO VASCULAR BARORECEPTORS.”

### INCORPORATION BY REFERENCE

All publications and patent applications mentioned in this specification are herein incorporated by reference to the same extent as if each individual publication or patent application was specifically and individually indicated to be incorporated by reference

### FIELD OF THE INVENTION

The devices and methods described herein relate generally to the treatment of hypertension.

### BACKGROUND OF THE INVENTION

Arterial hypertension, commonly referred to as “hypertension” or “high blood pressure”, is a medical condition in which the blood pressure is chronically elevated. Hypertension is associated with markedly elevated risk of heart attack, heart failure, arterial aneurysms, kidney failure and stroke. Causes of hypertension in a given individual may be one or more of many possibilities, which may include salt intake, obesity, occupation, alcohol intake, smoking, family size, stimulant intake, excessive noise exposure, and crowding, renin levels, insulin resistance, sleep apnea, genetic susceptibility, decreased kidney perfusion, catecholamine-secreting tumors of the adrenal glands, Adrenal hypertension with aldosterone-induced sodium retention, hypercalcemia, coarctation of the aorta, diet, medications, arterial stiffening that accompanies age. When the hypertension is secondary to another medical condition, it is generally prudent to treat that primary condition first. However, regardless as to whether the hypertension is primary or secondary, the blood pressure typically is subject to modification by several different approaches, including changing (typically via medications) fluid excretion, heart activity, and blood vessel contraction.

Medications for blood pressure control are frequently not effective, or present troublesome side effects when raised to a therapeutic dose. Depending on the class of medication, such side effects range from the inconvenient to the deadly, and may include constipation, edema, exercise intolerance, impotence, orthostasis, syncope and stroke.

Baroreceptors in the human body detect the pressure of blood flowing through them, and send messages to the central nervous system to increase or decrease total peripheral resistance and cardiac output, and thereby change blood pressure. There are baroreceptors in locations including the arch of the aorta, and the carotid sinuses of the left and right internal carotid arteries. Baroreceptors act to maintain mean arterial blood pressure to allow tissues to receive the right amount of blood. Neural signals from the baroreceptors are processed within the brain, in order to maintain physiological homeostasis. For example, the solitary nucleus and tract within the medulla and pons, receive signals from the carotid and aortic baroreceptors. In response to a perception of low blood pressure, the solitary nucleus sends out signals leading to hypertension, tachycardia and sympatho-excitation. In response to a perceived state of high blood pressure, the opposite physiological response is triggered.

Ultrasound is mechanical vibration at frequencies above the range of human hearing, or above 16 kHz. Most medical uses for ultrasound use frequencies in the range of 1 to 20 MHz. Low to medium intensity ultrasound products are widely used by physicians, nurses, physical therapists, masseurs and athletic trainers. The most common applications are probably warming stiff, swollen or painful joints or muscles in a manner similar to a hot compress, but with better penetration. Many ultrasound products have been commercially available for years, including consumer-grade massage machines. By design, the power on these devices is designed to be too low to warm or otherwise affect structures more than two centimeters or so below the surface. Also, these devices are not capable of tight focus at depth, nor are there means for accurately aiming such devices toward precise structural coordinates within the body. As ultrasound of sufficient strength can cause pain in peripheral nerves with each pulse, it is likely that mechanical perturbations caused by ultrasound can cause nerves to discharge. Similar effects may be produced by vibrations within the frequency range of human hearing (“sound”), and below the frequency range sensitivity of human hearing “sub-sound”.

Pulsed electrical currents are known to modify the function of nerves. At higher currents, this appears to be the result of direct nerve depolarization when the electrical gradient across a neural membrane is increased due to the passage of the electrical pulses. Examples of commercially available devices like this include the Activa deep brain stimulation system by Medtronic, Inc. Minneapolis, Minn. At lower currents, the firing thresholds of electrically excitable cells may be raised in response to steady sub-threshold stimulation. Examples of such devices include any of numerous commercially available transcutaneous electrical nerve stimulation (TENS) devices.

Static low-level direct electrical current has been shown to modify nerve function of both peripheral and central nerves. Unlike high current pulsatile forms of stimulation, the current does not directly drive action potentials. Instead, the flow of constant current between two distant poles modifies nerve function in ways that are not well understood, but which have been empirically documented. One example of such a technology is transcranial direct current stimulation (TDCS). It is hypothesized that membrane sensitivity and post-synaptic potentials are altered by the static presence of the electric field.

Pulsed magnetic fields are also known to modify nerve function in both peripheral and central nerves. Pulsed magnetic fields act chiefly by inducing electrical currents in conductive media through which the pulses pass. These transient induced electrical currents may serve to depolarize electrically excitable cells including neurons. Examples of pulsed magnetic fields that are known in the art include commercially available magnetic nerve stimulators such as the Magstim Rapid2 by Magstim Ltd, (Wales, UK).

Static magnetic fields may also modify nerve function, although the physiological mechanisms behind this approach are less clear. Static magnetic fields may influence the distribution of electrical charges on or within cellular membranes, as well as within areas of the electrically conductive cellular milieu. Protein folding and 3-configuration may also be influenced by static magnetic fields.

Baroreceptors are specialized nerve cells that serve to regulate blood pressure. They are found in the arch of the aorta, and in the carotid sinuses bilaterally, typically at and just distal to the carotid bifurcation. Baroreceptor cells are stretch-sensitive, and are increasingly activated as arteries expand under the force of blood pressure within that vessel. When blood pressure falls, for example, when a patient becomes dehydrated, baroreceptor-firing rate decreases. Signals from the carotid baroreceptors are sent through the glossopharyngeal nerve, and are relayed to the medulla, where they trigger reflexes that serve to lower blood pressure, for example by decreasing heart rate.

Massage or deep pressure to the carotid baroreceptors is a long-known method of abruptly lowering blood pressure. In recent years, surgically implantable neurostimulation devices have been developed to lower blood pressure by delivering pulsed electrical currents to the carotid baroreceptors (CVRx, Inc., Minneapolis, Minn.).

It would be desirable to non-invasively activate carotid baroreceptors using non-invasively delivered, benign and inexpensive energy forms such as ultrasound, magnetic pulses or by electrical current, so as to lower systemic blood pressure in hypertensive individuals.

### SUMMARY OF THE INVENTION

Described herein are methods and devices for treating hypertension by non-invasive techniques. In particular, described herein are devices and methods for treating hypertension by the application of non-invasively delivered energy to vascular baroreceptors (for example in the carotid sinuses of the neck). Delivery of this energy serves to lower blood pressure in hypertensive patients. For example, described herein are methods in which energy sources described in various embodiments include the use of sound, ultrasound, direct (DC) electrical current, pulsed electrical current, pulsed magnetic fields, and static magnetic fields. Delivery of the pulses is preferably accomplished through though patch-like transducers that are affixed to the skin, for example the upper anterior neck.

### BRIEF DESCRIPTION OF THE DRAWINGS

FIG. 1 shows a flow chart of the basic process steps involved with therapeutically using the present invention.

FIG. 2A illustrates an embodiment in which an ultrasonic transducer with an adhesive acoustic coupling layer serves to stimulate baroreceptor cells.

FIG. 2B illustrates the manner in which ultrasonic transducers are applied to the skin of the neck over each carotid sinus.

FIG. 3A illustrates a device for providing low-level direct electrical current stimulation through both carotid sinuses, using a bipolar pair of surface electrodes.

FIG. 3B illustrated the bipolar direct electrical current stimulation apparatus as applied to the neck of a patient.

FIG. 4 illustrates a generic embodiment of the present invention in which a shirt with collar can be used to conceal some or all of the apparatus.

FIG. 5A illustrates general aspects of an embodiment of the present invention in which electrical or magnetic stimulation is applied to the carotid baroreceptors via electrodes or magnetic coil applied to the surface of the skin overlying the carotid bifurcation.

FIG. 5B illustrates an embodiment of the present invention in which the stimulator is a static DC electrical field.

FIG. 5C illustrates an embodiment of the present invention in which the stimulator is a static magnetic field.

FIG. 5D illustrates an embodiment of the present invention in which the stimulator is an electrical pulse generator.

FIG. 5E illustrates an embodiment of the present invention in which the stimulator is a pulsed magnetic field generator.

### DETAILED DESCRIPTION OF THE INVENTION

The present invention is useful for enabling practical application of non-invasive stimulation of the arterial baroreceptors for the modification of blood pressure. In the context of this invention, the terms “sound”, “subsound” and “ultrasound” are used interchangeably. Additionally, the subsonic frequencies are subsumed under the term “sound”. While the present invention is not necessarily limited to such applications, various aspects of the invention may be appreciated through a discussion of various examples using this context. Various embodiments of the present invention are directed toward the use of ultra sound to produce carotid baroreceptor stimulation in a living subject. Sound waves are used to stimulate a first portion of neurons. Sound waves may also be used to generate electrical currents via specially designed devices. Such a device, if implanted surgically in proximity to a group of neurons that one wishes to affect, will serve to electrically stimulate those neurons when in receipt of sound waves. Additionally, magnetic coil stimulators may be surgically implanted adjacent to the carotid sinus, with the closer proximity of coil to baroreceptor serving to permit baroreceptor stimulation at lower power outputs from the stimulating device. While specific embodiments and applications thereof involve sound waves are described as being in the ultrasound frequency range, they need not be so limited. Aspects of the present invention employ frequencies outside of the ultrasound frequency range, including sonic and sub-sonic frequencies. In accordance with one embodiment, the present invention is directed to a method for modifying neural transmission patterns between neural structures. The method involves producing and directing sound waves toward a first targeted neural structure, controlling characteristics of the sound waves at the first target neural structure with respect to characteristics of sound waves. The present invention also regards use of low-level electrical current has been shown to modify nerve function. Unlike high current pulsatile forms of stimulation, the current does not directly drive action potentials. Instead, the flow of constant current between two distant poles modifies nerve function in ways that are not well understood, but which have been empirically documented. One example of such a technology is transcranial direct current stimulation (TDCS). Another suitable approach is pulsed electrical currents, for example that used in transcutaneous electrical nerve stimulation (TENS) units, which are commercially manufactured by numerous companies. It is hypothesized that membrane sensitivity and post-synaptic potentials are altered by the static presence of the electric field. Whether ultrasonic, magnetic or electrical stimulation is employed, the systems blood pressure is measured and the rate of stimulation applied is decreased or increased as appropriate to keep the systemic blood pressure in the desired range.

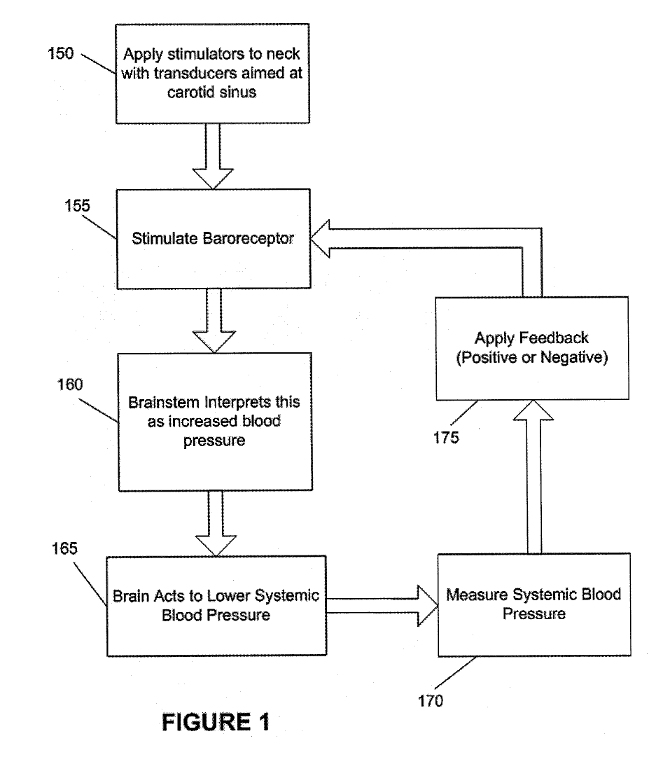

FIG. 1 shows a flow chart of the basic process steps involved with therapeutically using the present invention. In step 150, the stimulation transducers (for example, ultrasonic emitters, or, alternatively, electrodes) are applied to the skin of the neck, for example with an adhesive layer that serves to both conduct signal (acoustic coupling in the case of ultrasound, and electrical conduction in the case of electrodes), and hold the transducer in place. Other retaining means such as elastic straps may additionally be used to hold the transducers in place. In one embodiment, the transducers are built into the collar of a shirt, serving to both conceal the apparatus, as well as to hold the transducers in place. In step 155, the transducer is used to stimulate the carotid baroreceptors. In step 160, this baroreceptor stimulation is transmitted to the brainstem, which, in step 165, reflexively acts to lower systemic blood pressure. In step 170, the resulting systemic blood pressure is measured and in step 175, the stimulation of the baroreceptors is increased or decreased to keep the system blood pressure in the desired range.

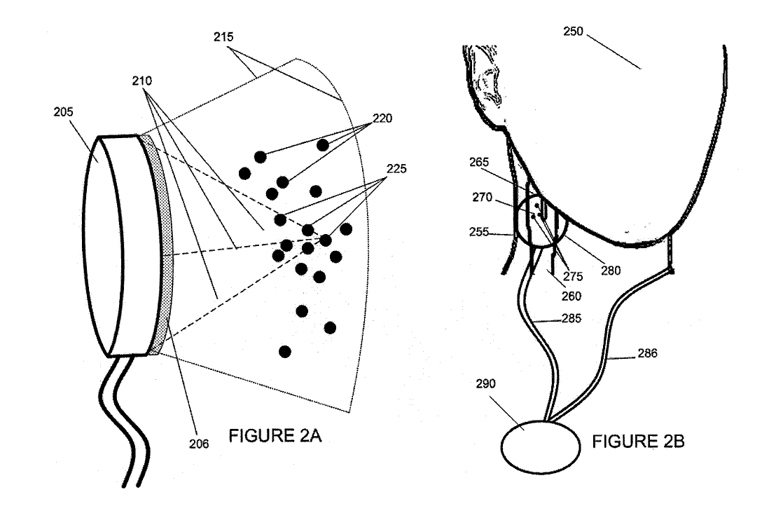

FIG. 2A illustrates an embodiment in which an ultrasonic transducer 205 which serves to stimulate baroreceptor cells, with an adhesive/acoustic coupling layer 206 which serves to both conduct signal (acoustic coupling in the case of ultrasound, and electrical conduction in the case of electrodes), and to stick to the skin and hold the transducer in place. Baroreceptors 220 and 225 (representative examples) are found within the carotid sinus (not shown). Transducer 205 may impart its energy broadly into the underlying tissue, fore example within the bounds of area 215, thereby stimulating both peripherally located baroreceptors 220 and centrally located baroreceptors 225. Transducer 205 may also be focused to impart its energy principally within lines 210, in which case 225, but not baroreceptors 220, will be stimulated. Because of the focused beam, a lower overall energy and associated power requirements may be feasible, but with the tradeoff of smaller area of stimulation.

FIG. 2B illustrates the manner in which ultrasonic transducer 280 and another on the opposite side of the neck (not shown) are applied to the skin of the neck 255 over the baroreceptors 275. Patient 250 has neck 255 with common carotid artery 260, which bifurcates into an external carotid artery (not labeled) and an internal carotid artery 265. Near the bifurcation within the proximal segment of the internal carotid artery, the vessel may enlarge somewhat in diameter, in an area known as the carotid sinus (270). Within the carotid sinus are specialized cells known as baroreceptors 275, which monitor blood pressure and insure appropriate delivery of blood to the brain. Pulse generator unit 290, via cord 285, powers ultrasound transducer 280. Pulse generator unit 290 via cord 286 powers a corresponding transducer on the opposite side of the neck.

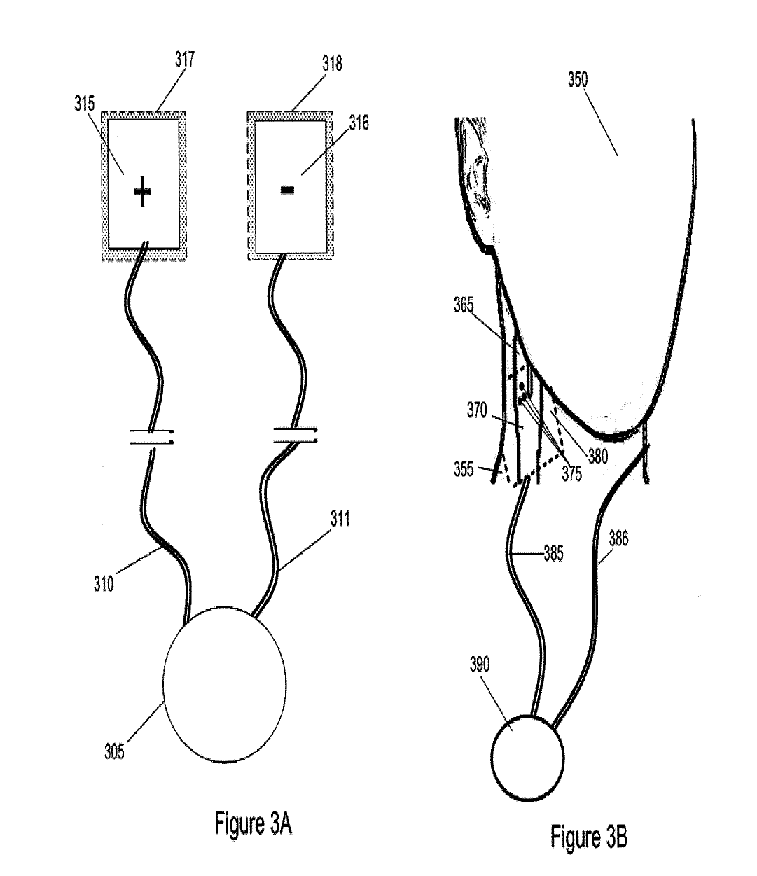

FIG. 3A illustrates a device for providing low-level direct electrical current stimulation through both carotid sinuses, using a bipolar pair of surface electrodes 315 and 316. Pulse generator unit has a positive output line 310 going to positive electrode 315, and a negative output line 311 going to negative electrode 316. Positive electrode 315 is physically adhered and electrically coupled to the skin of the neck by backing composite 317, and negative electrode 316 is physically adhered and electrically coupled to the skin on overlying the opposite carotid artery by backing composite 318. Backing composite 317 and 318 may be a combination of adhesive and electrical conduction substances in different areas, as is known in the art for the placement of various types of electrodes (for example electrocardiogram electrodes) on human skin. Typically, a waxed paper covering is peeled off to expose the adhesive and conductive surfaces before it is applied to cleansed skin.

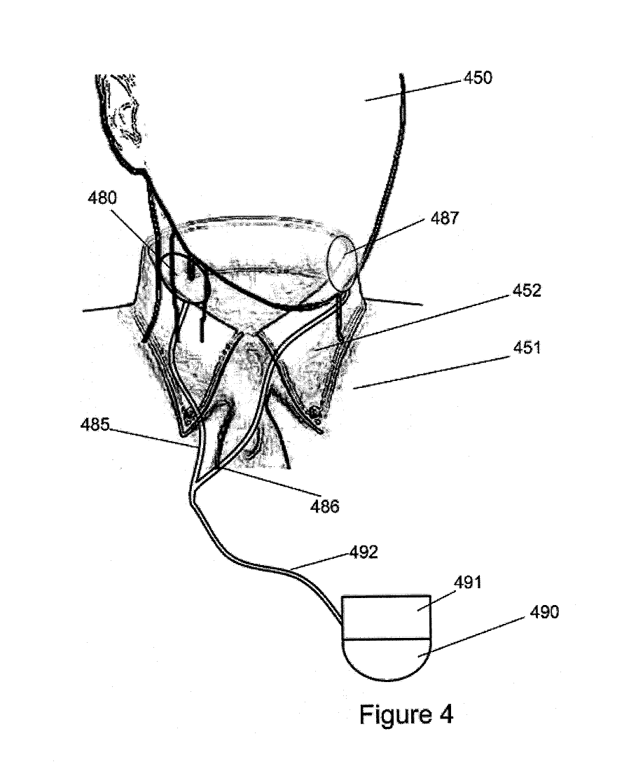

FIG. 3B illustrates the bipolar direct electrical current stimulation apparatus as applied to the neck 355 of patient 350. Internal carotid artery 370 branches into internal carotid artery 365 and external carotid artery (not labeled). Pulse generator unit 390 sends wire pair 385 to electrode 380, which overlies carotid baroreceptors 375. Corresponding wire pair 386 goes to an opposite-polarity electrode (not shown) on the opposite side of neck 355. FIG. 4 illustrates a generic embodiment of the present invention in which a patient 450 wears shirt 451 with collar 452 can be used to conceal some or all of the apparatus, as well as to physically hold them in place. Pulse generator unit 491 with battery portion 490 is connected via cable 492. Cable 492 bifurcates into a right cable branch 484 that serves transducer 480 and left cable branch 486 that serves transducer 487. Depending upon the height of collar 452, some or all of transducers 480 and 487 may be hidden. Transducers 480 and 487 may also be inserted within the folds of collar 452, rendering them invisible. Pulse generator unit 491 may be attached to a belt (not shown) or may be placed within a pocket (not shown) such as within shirt 45. Associated wire leads 492, 485 and 486 may be entirely beneath shirt 452.

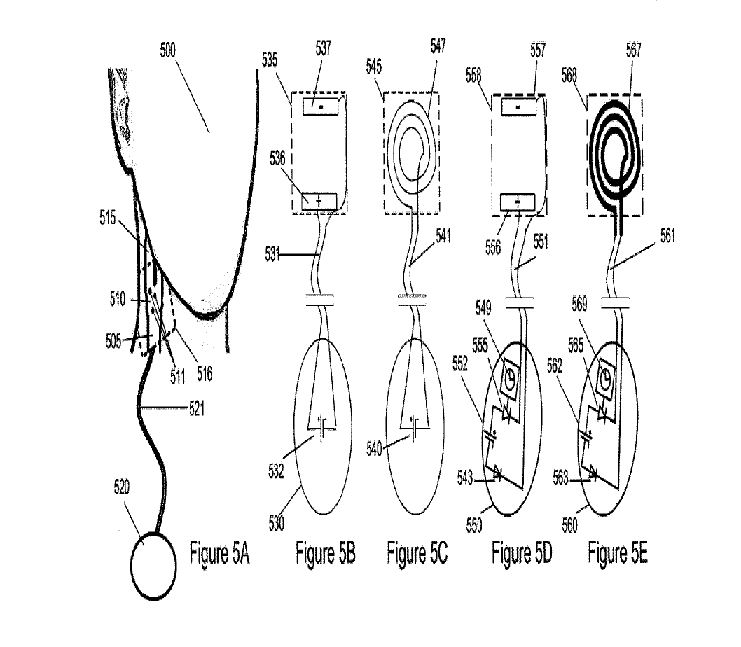

FIGS. 5A, 5B, 5C, 5D, and 5E illustrate the neuromodulation of the baroreceptors at the carotid bifurcation as a method of lowering blood pressure. In FIG. 5A, patient 500 has common carotid artery 515, internal carotid artery 515, and carotid bifurcation and sinus 510, containing carotid baroreceptor 511 (representative samples figuratively illustrated). On the surface of the skin overlying baroreceptors 511, the patient wears a patch 516, for example a flexible cloth-exterior dermal adhesive patch. In an alternative embodiment, this patch may be adhered to a buttoned shirt collar. On or more patches may be used, for example, one over the right carotid sinus, and one over the left carotid sinus. Attached to this patch are wire leads 531, and power source 520. Examples of the contents of the power source 520 and patch 516 are shown in FIGS. 5B, 5C, 5D and 5E. This power source 520 may be a “can” style enclosure like a pacemaker, but being non-implanted, is less constrained in terms of potential size. Power source 520 may reside in a shirt or jacket pocket, or may be clipped to a belt. A single power source may serve both a left and a right-sided patch 516.

In FIG. 5B, power supply 530 contains battery 532. A DC (direct) current flows from the poles of battery 532 through leads 531, to positive electrode 536 and negative electrode 537 on patch 535. This creates a DC current flow through the subcutaneous tissue beneath electrodes 537 and 536, including within the carotid sinus. Both electrodes (536 and 537) are attached to the underside of patch 538. Patch 538 may attach to the patient's skin with an adhesive, while the exposed surfaces of the electrodes 536 and 537 may be electrically couple to the skin with a conductive gel.

In FIG. 5C, power supply 540 contains battery 542. A DC (direct) current flows from the poles of battery 542 through leads 541, to electromagnetic coil 547 contained between the external layers of patch 545. This creates a steady magnetic field in the subcutaneous tissue below patch 545, including within the carotid sinus. Patch 548 may attach to the patient's skin with an adhesive.

In FIG. 5D, power supply 550 contains battery 552. Timed switch 549 may be a transistor, thyristor, gated diode or relay controlled by a time oscillator, timing chip or other time-control means known in the art. When timed switch 549 is momentarily closed, a current passes out from battery 552 into leads 551, in a phase controlled by diodes 455 and 553. These pulses of electrical current arrive at positive electrode 556 and negative electrode 557. Both electrodes (556 and 557) are attached to the underside of patch 558. Patch 558 may attach to the patient's skin with an adhesive, while the exposed surfaces of the electrodes 556 and 557 may be electrically couple to the skin with a conductive gel. This creates a pulsatile electrical flow through the subcutaneous tissue beneath electrodes 556 and 557, including within the carotid sinus.

In FIG. 5E, power supply 560 contains battery 562. Timed switch 569 may be a transistor, thyristor, gated diode or relay controlled by a time oscillator, timing chip or other time-control means known in the art. When timed switch 569 is momentarily closed, a current passes out from battery 562 into leads 561, in a phase controlled by diodes 565 and 563. These pulses of electrical current enter electromagnetic coil 567, which is held between the external layers of patch 465. The pulses of electrical current in electromagnetic coil 567 create a pulse magnetic field, in the subcutaneous tissue, which, in turn, induces a pulsed electrical field in the subcutaneous tissue beneath patch 568, including within the carotid sinus. Patch 558 may attach to the patient's skin with an adhesive. In each of the FIG. 5 embodiments, electrical fields or electrical currents in the carotid sinus serve to depolarize baroreceptor cells, causing them to relay a signal of excessive blood pressure to the brainstem.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. For instance, such changes may include variations in the duration and frequency of the stimulation between target areas.

### REFERENCES

“Device and Method for Treating Hypertension via Non-Invasive Neuromodulation” Partsch M J, Schneider, M. B. U.S. Patent application No. 60953191

DiLorenzo. U.S. Pat. No. 7,231,254. Jun. 12, 2007.

Kieval, Robert S., et al. U.S. Patent Application 20060293712, Dec. 28, 2006.

Prior Art Database. Citation IPCOM # 000156711D

U.S. Pat. No. 5,727,558

U.S. Pat. No. 6,050,953

U.S. Pat. No. 6,658,298

U.S. Pat. No. 6,393,324

U.S. Pat. No. 6,178,352

U.S. Patent Application No. 20040102818

What is claimed is:

1. A device for modifying blood pressure comprising:
one or more energy emitters;
control means for regulating the output of said emitter; and
means for fastening said emitter against skin overlying the carotid sinus whereby output of said emitters stimulates the carotid baroreceptors causing reflex lowering of blood pressure.

2. A device as in claim 1 in which said energy emitted is sound

3. A device as in claim 1 in which said energy emitted is ultrasound

4. A device as in claim 1 in which said energy emitted is subsonic.

5. A device as in claim 1 in which said energy emitted is a direct electrical current

6. A device as in claim 1 in which said energy emitted is a pulsed electrical current

7. A device as in claim 1 in which said energy emitted is a static magnetic field

8. A device as in claim 1 in which said energy emitted is a pulsed magnetic field

9. A device as in claim 1 also comprising feedback means for relaying measured blood pressure back to said control means whereby output stimulation from said emitters is modified by said blood pressure measurement and blood pressure modulated up and down accordingly.

10. A device for modifying blood pressure comprising:
one or more electrode pads;
an electrical power source;
control means for regulating the output of said power source; and
means for fastening said electrode pads against skin overlying the carotid sinus.

11. A device as in claim 1 also comprising feedback means for relaying measured blood pressure back to said control means whereby output stimulation from said emitters is modified by said blood pressure measurement.

12. A method for lowering blood pressure comprising:
directing the transcutaneous flow of energy toward carotid baroreceptors so as to stimulate said baroreceptors;
a device as in claim 1 wherein said device is an adhesive patch.
